(19) United States
(12) Patent Application Publication
(10) Pub. No.: US 2006/0083724 A1
(43) Pub. Date: Apr. 20, 2006

(54) Compositions for the treatment of femal sexual dysfunction

(76) Inventors: Todd W. Hilst, Littleton, CO (US); Bonita L. Griffin Kaake, Lakewood, CO (US); Heidi Kristine Meyer, Tabernash, CO (US)

Correspondence Address: THE MCCALLUM LAW FIRM, LLC, 132 KOLAR COURT, ERIE, CO, 80516, US

(21) Appl. No.: 11/243,207

(22) Filed: Oct. 3, 2005

Related U.S. Application Data
(60) Provisional application No. 60/615,330, filed on Oct. 1, 2004.

(51) Int. Cl. A61K 35/74; A61K 38/09; A61K 36/534; A61K 36/82; A61K 31/522; A61K 31/455

(52) U.S. Cl. 424/93.45; 424/747; 514/263.34; 514/355; 514/565; 514/724; 514/643; 514/15; 514/29; 514/35

(57) ABSTRACT

The compositions and methods disclosed herein are of use for the treatment of female sexual dysfunction. The disclosed compositions and methods pertain to topical formulations including at least three vasodilating agents that act by different physiological pathways, and at least one sensory or functional additive.

### RELATED APPLICATIONS

The present application claims the benefit of U.S. Provisional Patent Application No. 60/615,330, filed Oct. 1, 2004, which is hereby incorporated by reference in its entirety.

### BACKGROUND OF THE INVENTION

1. Field of the Invention

The present invention relates to compositions and methods for increasing sexual response and ameliorating or preventing the affects of female sexual dysfunction; and, specifically to the use of topical formulations that create more satisfactory sexual experiences for both men and women.

2. Background

Female sexual dysfunction is generally defined as a persistent or recurrent problem related to sexual response. Such problems may include loss of sexual desire or arousal, pain or discomfort during intercourse (dyspareunia), diminished blood flow to the vagina and genital area, inability to achieve orgasm and general sexual dissatisfaction. There are numerous factors that contribute to sexual dysfunction; for example, physiological problems, hormonal imbalances, prescribed medications (like antidepressants), infections, diseases (like diabetes or multiple sclerosis) that have potential side effects such as pain during sex or excessive dryness, vascular conditions, pregnancy, childbirth, menopause and the stress of daily life. A number of these contributing factors are on the rise within the American population (e.g., diabetes, use of antidepressants, and hormonal imbalances associated with cessation of hormone replacement therapy, which recent studies have linked to cancer).

Additionally, an aging population creates increasing numbers of women who will be entering menopause in the near future. Menopause is accompanied by various physiological changes that can affect sexual function. For example, estrogen deficiency leads to loss of skin elasticity (wrinkles), which presents itself in the genitalia as shrinking and thinning of the vagina and labia. The vagina becomes narrower and the natural swelling and lubrication that occurs during the arousal phase of intercourse can take longer. As a result intercourse can be difficult, painful and/or frustrating. In the labia, loss of skin elasticity leads to increased exposure of the clitoris that can lead to reduced sensitivity of the clitoris and/or an unpleasant tingling or prickling sensation.

A formulation capable of increasing female sexual response and ameliorating or preventing the affects of female sexual dysfunction would create more satisfactory sexual experiences for both men and women.

### DETAILED DESCRIPTION

In accordance with the purpose(s) of this invention, as embodied and broadly described herein, this invention, in one aspect, relates to compositions and methods for increasing sexual response in a female.

Particular advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

Definitions

For the purposes of the present invention, the following terms shall have the following meanings:

For the purposes of the present invention, ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

For the purposes of the present invention, the term “increased sexual response” shall refer to physiological or psychological conditions that improve a woman's preparedness for intercourse. Exemplary “increased sexual responses” include ease of arousal, increased desire, increased production of vaginal secretions, decreased pain, increased intensity of or number of orgasms, decreased time to reach orgasm or the like.

For the purposes of the present invention, the term “sensory additive” or “sensory agent” shall refer to a composition that affects the senses of a woman and/or her sexual partner. For example, a sensory agent may affect sight, smell, taste, touch or hearing.

For the purposes of the present invention, the term “functional additive” or “functional agent” shall refer to a composition that serves a purpose other than to stimulate the senses. Functional additives may, for example, help to prevent infections (including transmission of sexually transmitted diseases), improve skin tone and elasticity or prevent or aid in pregnancy, for example. Additives that act as pregnancy aids are referred to herein as “fertility agents”. In a particular embodiment, a fertility agent sustains or improves the viability of sperm.

For the purposes of the present invention, the term “microbicide” shall refer to any substance that can substantially reduce transmission of sexually transmitted infections (STIs) when applied to the genitalia such that the microbicide comes into contact with sexual fluids.

For the purposes of the present invention, the term “spermicide” shall refer to a composition that kills or disables sperm so that it cannot cause pregnancy.

Finally, for the purposes of the present invention, the terms “individual”, “patient”, and “subject” are to mean a female human.

Moreover, for the purposes of the present invention, the term “a”, “an” or “the” refers to one or more of that entity; for example, “a blocker” or “an inhibitor” refers to one or more of those elements or at least one element. As such, the terms “a” or “an”, “one or more” and “at least one” can be used interchangeably herein. It is also to be noted that the terms “comprising,” “including,” and “having” can be used interchangeably. Furthermore, an element “selected from the group consisting of” refers to one or more of the elements in the list that follows, including mixtures (i.e. combinations) of two or more of the elements.

Reference will now be made in detail to particular embodiments of the invention.

The present invention embodies compositions and methods related to novel treatments for female sexual dysfunction. In one embodiment, a method of the present invention includes applying a topical formulation including at least three vasodilating agents to the clitoris of a female, where the three vasodilating agents act by different physiological pathways, and at least one sensory or functional additive.

According to an embodiment of the present invention, a topical formulation includes a phosphodiesterase (PDE) inhibitor, a calcium channel blocker and a NO donor agent. In a particular embodiment, the phosphodiesterase inhibitor is theopylline or aminophylline, the calcium channel blocker is nifedipine, and the NO donor is L-arginine. In another particular embodiment, peppermint oil may be added to the composition to improve smell and taste.

In a certain embodiment, a composition of the present invention comprises about 0.1-0.3% nifedipine, about 3-5% aminophylline and about 8-10% arginine. In a particular embodiment, a composition of the present invention comprises about 0.15% nifedipine, about 4.5% aminophylline, about 9% arginine and about 1% glycerine formulated in a base of PCCA vanishing cream lite.

In a particular embodiment, compositions of the present invention include at least three vasodilating agents, each acting by a different physiological pathway, and at least one additive selected from the group consisting of a topical antibiotic, a skin toning agent, a microbicide, a spermicide, a fertility agent, a smell agent, a taste agent, a coloring agent, a warming agent, a cooling agent, and a variable temperature agent. Exemplary warming, cooling and variable temperature agents include capsaisin, menthol and methyl salicylate.

In a certain embodiment, a microbicide is selected from the group consisting of C31G, Carbopol 974P, Carrageenan, Cyanovirin-N, Hydroxyethyl cellulose, PRO 2000, UC-781, Cellulose Sulfate, Dextrin-2-sulfate, PC515, BufferGel, Lactobacillus crispatus, Praneem, Tenofovir disoproxil fumarate and similar detergents and surfactants. In another embodiment, a spermicide is selected from the group consisting of nonoxynol-9, oxtoxynol-9, chlorhexidine, and benzalkonium chloride. In yet another embodiment, a fertility agent is selected from the group consisting of Gonadotropin Releasing Hormone, Luteinizing Hormone, Follicle Stimulating Hormone, Clomid (Clomiphene citrate), and Parlodel (bromocriptine mesylate).

In a certain embodiment, a topical antibiotic is selected from the group consisting of bacitracin, neomycin, mupirocin, erythromycin, clindamycin, polymyxin B, sulfanilamide, clotrimazole, metronidazole, ampicilin, mycostatin, ciprofloxacin and the like. The presence of a topical antibiotic in a formulation of the present invention may facilitate healing of micro tears or abrasions resulting from friction associated with sexual intercourse. As open wounds are known to increase the risk of transmission of sexually transmitted diseases (STDs), a composition that facilitates rapid healing may help to decrease the incidence of STDs.

In another particular embodiment, a skin toning agent may be selected from the group consisting of vitamin A (retinol), vitamin C, vitamin E, N-6 furfuryladenine (kinetin), green tea extract, cocoa butter, aloe vera, shea butter, lanolin, marine collagens, and other compounds known to improve skin tone or elasticity. All skin toning agents known to dermatologists, cosmetologists, or aestheticians are contemplated for use in formulations of the present invention with the exclusion of those which are incompatible with the genital environment. As described above, female sexual dysfunction is often related to physiological changes occurring to the genitalia with age. In particular, loss of skin elasticity results in thinning of the vagina and labia and increased exposure of the clitoris. Therefore, the application of a pharmaceutical composition comprising a skin toning agent would help to halt or reverse the effects of aging on the genitalia, thus eliminating at least one of the factors contributing to female sexual dysfunction.

In another embodiment, the compositions and methods of the present invention do not create adverse effects such as burning, itching, allergic reaction or infection.

Pharmaceutical Formulations

Formulations of the present invention may be administered by transdermal or transmucosal administration to the female genitalia. The terms “transdermal” and “transmucosal” are used interchangeably herein to refer to passage through various membranes which may include, but are not limited to, clitoral, vaginal and labial membranes. All such methods of administration are well known in the art. Compositions of the present invention are applied topically. Such compositions include, but are not limited to, lotions, ointments, creams, gels, drops, sprays, liquids and powders. Conventional pharmaceutical carriers, aqueous, powder or oily bases, thickeners, transdermal delivery agents and the like may be necessary or desirable. For example, vanishing cream is a commonly used base, glycerine is a well known wetting agent, and N,N-dimethylsulfoxide (DMSO) and N,N-dimethylformamide (DMF) are known transdermal delivery agents.

Antimicrobial preservatives and appropriate drug stabilizers, if required, may be included in any of the formulations. Preservatives and other additives or preservation techniques may be selected from the group consisting of, but not limited to, antimicrobials, anti-oxidants, chelating agents, inert gases, vacuum packing procedures and the like. Various commercial chemicals are sold with preservatives included.

In a particular embodiment, compositions of the present invention are designed to be administered topically to the genitalia, and typically to the clitoris, of a female from about 0-90 minutes prior to intercourse or masturbation. The amount to be applied will vary from woman to woman depending on physiological factors and personal preferences.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the invention.

### Example 1

### Clinical Trial of Three Formulations

Thirty female patients 40-60 years of age enrolled in a voluntary trial. The women were given a tube containing a topical cream of an over-the-counter product to be applied to the female genitalia, specifically to the clitoris, prior to sexual activity. After 30 days of use, they were given a second tube of topical cream comprising a composition of the present invention and a questionnaire to assess their relative impressions of the products.

The questionnaire addressed the following:

Compared to the first product you sampled, which items below describe the difference between this product and the first one you sampled?

| | |
|---|---|
| Decreased foreplay duration? | 3 |
| Change in quantity of orgasms? | 3 |
| Change is quality of orgasms? | |
| Better? | 4 |
| Worse? | 0 |
| Increased moisture/warmth? | 10 |
| Increased desire? | 6 |

Would you continue to use this product after the test phase is over?

| | |
|---|---|
| Yes | 10 |
| No | 3 |

Would you recommend this product formulation over the other?

| | |
|---|---|
| Yes | 9 |
| No | 2 |

In your opinion, compared to the second sample, how much more effective is the second formulation?

Thirteen female volunteers reported on the effectiveness of both the composition of the present invention and a typical over-the-counter (OTC) product. Four volunteers reported that the present composition was 100% more effective than the OTC product; 1 volunteer reported that the present composition was 75% more effective than the OTC product; 5 volunteers reported that the present composition was 50% more effective than the OTC product; 2 volunteers reported that the present composition was 25% more effective than the OTC product; and 1 volunteer reported that the present composition was not as effective as the OTC product.

All of the COMPOSITIONS, METHODS and APPARATUS disclosed and claimed herein can be made and executed without undue experimentation in light of the present disclosure. While the methods and apparatus of this invention have been described in terms of preferred embodiments, it will be apparent to those of skill in the art that variations may be applied to the COMPOSITIONS, METHODS and APPARATUS and in the steps or in the sequence of steps of the methods described herein without departing from the concept, spirit and scope of the invention. More specifically, it will be apparent that certain related components may be substituted for the components described herein while the same or similar results would be achieved. All such similar substitutes and modifications apparent to those skilled in the art are deemed to be within the spirit, scope and concept of the invention as defined by the appended claims.

What is claimed is:

1. A composition comprising at least three vasodilating agents and at least one additive selected from the group consisting of a sensory additive and a functional additive, wherein said vasodilating agents each act by a different physiological pathway.

2. The composition of claim 1, wherein said at least three vasodilating agents are aminophylline, arginine and nifedipine.

3. The composition of claim 1, wherein said sensory additive is selected from the group consisting of a smell agent, a taste agent, a warming agent, a cooling agent, and a variable temperature agent.

4. The composition of claim 3, wherein said composition comprises aminophylline, arginine, nifedipine and peppermint oil.

5. The composition of claim 1, wherein said functional additive is selected from the group consisting of a topical antibiotic, a skin toning agent, a microbicide, a spermicide and a fertility agent.

6. The composition of claim 5, wherein said composition comprises aminophylline, arginine, nifedipine and a spermicide selected from the group consisting of nonoxynol-9, oxtoxynol-9, chlorhexidine and benzalkonium chloride.

7. The composition of claim 5, wherein said composition comprises aminophylline, arginine, nifedipine and a microbicide selected from the group consisting of C31G, Carbopol 974P, Carrageenan, Cyanovirin-N, Hydroxyethyl cellulose, PRO 2000, UC-781, Cellulose Sulfate, Dextrin-2-sulfate, PC515, BufferGel, Lactobacillus crispatus, Praneem and Tenofovir disoproxil fumarate.

8. The composition of claim 5, wherein said composition comprises aminophylline, arginine, nifedipine and a fertility agent selected from the group consisting of Gonadotropin Releasing Hormone, Luteinizing Hormone, Follicle Stimulating Hormone, Clomid (Clomiphene citrate) and Parlodel (bromocriptine mesylate).

9. The composition of claim 5, wherein said composition comprises aminophylline, arginine, nifedipine and a topical antibiotic is selected from the group consisting of bacitracin, neomycin, mupirocin, erythromycin, clindamycin, polymyxin B, sulfanilamide, clotrimazole, metronidazole, ampicilin, mycostatin and ciprofloxacin.

10. The composition of claim 5, wherein said composition comprises aminophylline, arginine, nifedipine and a skin toning agent selected from the group consisting of vitamin A (retinol), vitamin C, vitamin E, N-6 furfuryladenine (kinetin), green tea extract, cocoa butter, aloe vera, shea butter, lanolin and marine collagens.

11. A composition comprising about 0.1-0.3% nifedipine, about 3-5% aminophylline and about 8-10% arginine.

12. The composition of claim 11, comprising about 0.15% nifedipine, about 4.5% aminophylline, about 9% arginine and about 1% glycerine formulated in a base of vanishing cream.

13. The composition of claim 11, further comprising peppermint oil.

14. The composition of claim 11, further comprising a functional additive selected from the group consisting of a topical antibiotic, a skin toning agent, a microbicide, a spermicide and a fertility agent.

15. The composition of claim 14, wherein said composition further comprises a spermicide selected from the group consisting of nonoxynol-9, oxtoxynol-9, chlorhexidine and benzalkonium chloride.

16. The composition of claim 14, wherein said composition further comprises a microbicide selected from the group consisting of C31G, Carbopol 974P, Carrageenan, Cyanovirin-N, Hydroxyethyl cellulose, PRO 2000, UC-781, Cellulose Sulfate, Dextrin-2-sulfate, PC515, BufferGel, Lactobacillus crispatus, Praneem and Tenofovir disoproxil fumarate.

17. The composition of claim 14, wherein said composition further comprises a fertility agent selected from the group consisting of Gonadotropin Releasing Hormone, Luteinizing Hormone, Follicle Stimulating Hormone, Clomid (Clomiphene citrate) and Parlodel (bromocriptine mesylate).

18. The composition of claim 14, wherein said composition further comprises a topical antibiotic is selected from the group consisting of bacitracin, neomycin, mupirocin, erythromycin, clindamycin, polymyxin B, sulfanilamide, clotrimazole, metronidazole, ampicilin, mycostatin and ciprofloxacin.

19. The composition of claim 14, wherein said composition further comprises a skin toning agent selected from the group consisting of vitamin A (retinol), vitamin C, vitamin E, N-6 furfuryladenine (kinetin), green tea extract, cocoa butter, aloe vera, shea butter, lanolin and marine collagens.

20. A method of treating female sexual dysfunction comprising topically applying, to the clitoris of a female in need of such treatment, a composition comprising at least three vasodilating agents and at least one additive selected from the group consisting of a sensory additive and a functional additive, wherein said vasodilating agents each act by a different physiological pathway.

21. The method of claim 20, wherein said at least three vasodilating agents are aminophylline, arginine and nifedipine.

22. The method of claim 20, wherein said sensory additive is selected from the group consisting of a smell agent, a taste agent, a warming agent, a cooling agent, and a variable temperature agent.

23. The method of claim 22, wherein said composition comprises aminophylline, arginine, nifedipine and peppermint oil.

24. The method of claim 20, wherein said functional additive is selected from the group consisting of a topical antibiotic, a skin toning agent, a microbicide, a spermicide and a fertility agent.

25. The method of claim 24, wherein said composition comprises aminophylline, arginine, nifedipine and a spermicide selected from the group consisting of nonoxynol-9, oxtoxynol-9, chlorhexidine and benzalkonium chloride.

26. The method of claim 24, wherein said composition comprises aminophylline, arginine, nifedipine and a microbicide selected from the group consisting of C31G, Carbopol 974P, Carrageenan, Cyanovirin-N, Hydroxyethyl cellulose, PRO 2000, UC-781, Cellulose Sulfate, Dextrin-2-sulfate, PC515, BufferGel, Lactobacillus crispatus, Praneem and Tenofovir disoproxil fumarate.

27. The method of claim 24, wherein said composition comprises aminophylline, arginine, nifedipine and a fertility agent selected from the group consisting of Gonadotropin Releasing Hormone, Luteinizing Hormone, Follicle Stimulating Hormone, Clomid (Clomiphene citrate) and Parlodel (bromocriptine mesylate).

28. The method of claim 24, wherein said composition comprises aminophylline, arginine, nifedipine and a topical antibiotic is selected from the group consisting of bacitracin, neomycin, mupirocin, erythromycin, clindamycin, polymyxin B, sulfanilamide, clotrimazole, metronidazole, ampicilin, mycostatin and ciprofloxacin.

29. The method of claim 24, wherein said composition comprises aminophylline, arginine, nifedipine and a skin toning agent selected from the group consisting of vitamin A (retinol), vitamin C, vitamin E, N-6 furfuryladenine (kinetin), green tea extract, cocoa butter, aloe vera, shea butter, lanolin and marine collagens.

30. The method of claim 20, wherein said composition comprises about 0.1-0.3% nifedipine, about 3-5% aminophylline and about 8-10% arginine.

31. The method of claim 30, wherein said composition comprises about 0.15% nifedipine, about 4.5% aminophylline, about 9% arginine and about 1% glycerine formulated in a base of vanishing cream.
